(19) United States
(12) Patent Application Publication
(10) Pub. No.: US 2003/0036857 A1
(43) Pub. Date: Feb. 20, 2003

(54) Methods and systems of biomolecular sequence matching

(76) Inventors: Xiang Yao, San Diego, CA (US); Heng Dai, San Diego, CA (US); Albert Leung, San Diego, CA (US); Bin Tian, San Diego, CA (US); Wei D. Zhao, San Diego, CA (US); Xuejun Liu, San Diego, CA (US); Joseph Ciervo, San Diego, CA (US); Simon R. Smith, San Diego, CA (US); Jackson S. Wan, San Diego, CA (US)

Correspondence Address: McKenna Long & Aldridge LLP, 1900 K Street, N.W., Washington, DC, 20006, US

(21) Appl. No.: 10/209,660

(22) Filed: Aug. 1, 2002

Related U.S. Application Data
(60) Provisional application No. 60/308,839, filed on Aug. 1, 2001.

(51) Int. Cl. G06F 19/00; G01N 33/48; G01N 33/50

(52) U.S. Cl. 702/20

(57) ABSTRACT

The present invention relates to methods and systems for database comparison and database searching and matching, and specifically to database comparison and database searching and matching of databases containing biomolecular sequences as well as databases comprising matched sequences, as well as the use of databases comprising matched biomolecular sequences. In addition, a database comprising matched sequences or a database comprising matched biomolecular sequences may be accessed via a graphical user interface.

### FIELD OF THE INVENTION

The present invention relates to methods and systems for database comparison and database searching and matching, and specifically to database comparison and database searching and matching of databases containing biomolecular sequences as well as databases comprising matched sequences, as well as the use of databases comprising matched biomolecular sequences.

### BACKGROUND OF THE INVENTION

The number of known and identified human DNA sequences is only a small fraction of the enormous total number of human DNA sequence combinations, and the number of such known and identified DNA sequences is growing rapidly. In addition, the number of DNA sequences of other organisms that have been identified and that are available in databases is also large and likewise growing with time.

The DNA sequence information contained in these growing databases will be a major instrument for basic medical and biological research activities for many years. This information will also be a basis for developing curative techniques for medical and hereditary afflictions. In order to use effectively the information in these enormous and growing databases, it is necessary to provide an efficient means to access and manipulate that information. In particular, it is necessary to provide an efficient and reliable means to compare a given DNA sequence to the library of known DNA sequences in the databases. Such a comparison is useful to identify, analyze, and interpret that given DNA sequence.

Current procedures for making such comparisons are comparatively slow and impractical. As the amount of stored information increases, current search methods will become unable to function with practical, short processing times, and these methods will have very slow operating speeds. Thus, there is an important and immediate need for systems and procedures to perform DNA sequence matching with convenient database access, high speed processing, accuracy, and cost efficiency.

Additionally, the rapid growth of available high quality DNA sequence data has made mass spectrometry (MS) combined with genome database searching a popular and potentially accurate method to identify proteins. Protein identification by mass spectrometry has proven to be a powerful tool to elucidate biological function and to find the composition of protein complexes and entire organelles.

The relationship between structure and function of macromolecules is of fundamental importance in the understanding of biological systems. These relationships are important to understanding, for example, the functions of enzymes, structural proteins, and signaling proteins, ways in which cells communicate with each other, as well as mechanisms of cellular control and metabolic feedback.

There are various algorithms that attempt to identify the protein with the highest degree of similarity to the experimentally obtained peptide map. Methods for evaluating the quality of a protein identification result have recently been provided. However, such methods may be computationally intensive, may not always be readily integrated with search programs and may need to set different standards for different databases. As increasingly complex biological problems are explored, simplified methods to evaluate the quality of a protein identification result are critical.

This invention generally relates to methods and systems for analyzing data, and more particularly to methods and systems for searching databases for a given record. More specifically, the present invention relates to a methods and systems for searching a database of known biomolecular sequences for a biomolecular sequence that matches or closely resembles a given biomolecular sequence.

### SUMMARY OF THE INVENTION

The present invention is directed to methods and systems for database comparison and database searching and matching, and specifically to methods and systems of database comparison and database searching and matching of databases containing biomolecular sequences as well as databases comprising matched sequences, as well as the use of databases comprising matched biomolecular sequences.

In a specific embodiment, the present invention may comprise a method of comparing information contained in biomolecular databases by matching sequence identification information of biomolecular sequences from two databases and placing any matching biomolecular sequences in a matched sequence database. In a specific embodiment, matched biomolecular sequences are removed from the databases being compared. In the specific embodiment, the method may further involve matching biomolecular sequence information of biomolecular sequences from one database with clusters of biomolecular sequences in another database and placing any matching biomolecular sequences in the matched sequence database. In a specific embodiment, the biomolecular sequence information may be matched with portions of a consensus or contig biomolecular sequence of biomolecular sequences. In a specific embodiment, biomolecular sequences matched with clusters of biomolecular sequences are removed from the first database. In the specific embodiment, the method may further involve matching complete biomolecular sequence information of biomolecular sequences from two databases and placing any matching biomolecular sequences in the matched sequence database. In a specific embodiment, one of the databases being compared may be an internal database including, but not limited to, Incyte, DNAchip Memo Status, Gene Expression, PRI Classification, and Proteome. In a specific embodiment, one of the databases being compared may be an external database including, but not limited to, InterPro, Ensembl, dbSNP, OMIM, LocusLink, GeneOntology, UniGene, and HomoloGene. In a specific embodiment, one or more of the databases are clustered before being matched. In a specific embodiment, the matching may be done when one or more of the databases is updated.

In a specific embodiment, a matched sequence database is obtained from the methods described above.

In a specific embodiment, a system for producing a matched sequence database matches sequence identification information of biomolecular sequences from two databases and places any matching biomolecular sequences in a matched sequence database. In a specific embodiment, matched biomolecular sequences are removed from the databases being compared. In the specific embodiment, the system may further involve matching biomolecular sequence information of biomolecular sequences from one database with clusters of biomolecular sequences in another database and placing any matching biomolecular sequences in the matched sequence database. In a specific embodiment, the biomolecular sequence information may be matched with portions of a consensus or contig biomolecular sequence of biomolecular sequences. In a specific embodiment, biomolecular sequences matched with clusters of biomolecular sequences are removed from the first database. In the specific embodiment, the system may further involve matching complete biomolecular sequence information of biomolecular sequences from two databases and placing any matching biomolecular sequences in the matched sequence database. In a specific embodiment, one of the databases being compared may be an internal database including, but not limited to, Incyte, DNAchip Memo Status, Gene Expression, PRI Classification, and Proteome. In a specific embodiment, one of the databases being compared may be an external database including, but not limited to, InterPro, Ensembl, dbSNP, OMIM, LocusLink, GeneOntology, UniGene, and HomoloGene. In a specific embodiment, one or more of the databases are clustered before being matched. In a specific embodiment, the matching may be done when one or more of the databases is updated.

In a specific embodiment, a method for constructing a matched sequence database in a computer system may compare information contained in biomolecular databases by matching sequence identification information of biomolecular sequences from two databases and place any matching biomolecular sequences in a matched sequence database. In a specific embodiment, matched biomolecular sequences are removed from the databases being compared. In the specific embodiment, the method in a computer system may further consist of matching biomolecular sequence information of biomolecular sequences from one database with clusters of biomolecular sequences in another database and placing any matching biomolecular sequences in the matched sequence database. In a specific embodiment, the biomolecular sequence information may be matched with portions of a consensus or contig biomolecular sequence of biomolecular sequences. In a specific embodiment, biomolecular sequences matched with clusters of biomolecular sequences are removed from the first database. In the specific embodiment, the method in a computer system may further consist of matching complete biomolecular sequence information of biomolecular sequences from two databases and placing any matching biomolecular sequences in the matched sequence database. In a specific embodiment, one of the databases being compared may be an internal database including, but not limited to, Incyte, DNAchip Memo Status, Gene Expression, PRI Classification, and Proteome. In a specific embodiment, one of the databases being compared may be an external database including, but not limited to, InterPro, Ensembl, dbSNP, OMIM, LocusLink, GeneOntology, UniGene, and HomoloGene. In a specific embodiment, one or more of the databases are clustered before being matched. In a specific embodiment, the matching may be done when one or more of the databases is updated.

In a specific embodiment, a computer program may be used to construct a matched sequence database by implementing a first module adapted to match biomolecular sequence information. In a specific embodiment the first module may comprise an algorithm for matching sequence identification information of biomolecular sequences from two databases and placing any matching biomolecular sequences in a matched sequence database. In a specific embodiment, matched biomolecular sequences are removed from the databases being compared. In the specific embodiment, the computer program may be further implemented a second module adapted to match biomolecular sequence information. In a specific embodiment, the second module may comprise an algorithm for matching biomolecular sequence information of biomolecular sequences from one database with clusters of biomolecular sequences in another database and placing any matching biomolecular sequences in the matched sequence database. In a specific embodiment, the biomolecular sequence information may be matched with portions of a consensus or contig biomolecular sequence of biomolecular sequences. In a specific embodiment, biomolecular sequences matched with clusters of biomolecular sequences are removed from the first database. In the specific embodiment, the computer program may be further implemented by a third module adapted to match biomolecular sequence information. In a specific embodiment, the third module may comprise an algorithm for matching complete biomolecular sequence information of biomolecular sequences from two databases and placing any matching biomolecular sequences in the matched sequence database. In a specific embodiment, one of the databases being compared may be an internal database including, but not limited to, Incyte, DNAchip Memo Status, Gene Expression, PRI Classification, and Proteome. In a specific embodiment, one of the databases being compared may be an external database including, but not limited to, InterPro, Ensembl, dbSNP, OMIM, LocusLink, GeneOntology, UniGene, and HomoloGene. In a specific embodiment, one or more of the databases are clustered before being matched. In a specific embodiment, the matching may be done when one or more of the databases is updated.

In a specific embodiment of the present invention, a computer system may provide users with the ability to access biomolecular sequence information from a matched sequence database. In a specific embodiment, the computer system may consist of a computer processor, a memory operatively coupled to the computer processor, and the computer program described above stored in the memory.

In a specific embodiment of the present invention, a computer process may allow a user to interactively access biomolecular sequence information from the matched sequence database by providing a graphical user interface containing query options for the search and displaying the results. In a specific embodiment, the query options may include, but are not limited to, one or more of selecting one or more biomolecular sequences, one or more external databases from which information related to the biomolecular sequences being searched may be extracted, and one or more fields within each external database.

In a specific embodiment of the present invention, a method of accessing and displaying biomolecular sequence information from a matched sequence database may include selecting one or more biomolecular sequences, one or more fields of the matched sequence database, and performing a database query on the one or more fields of the matched sequence database for the one or more biomolecular sequences.

In a specific embodiment, a business method may consist of providing a matched sequence database to a consumer. In a specific embodiment, the business method may further consist of charging a fee to the consumer for providing the database. In a further embodiment, the fee may be charged to the consumer by selling a license, charging a per-access fee to the database, or charging a time-based fee for accessing the database.

In a specific embodiment, a business method may consist of providing a matched sequence database to a third party vendor.

In a specific embodiment, a business method may consist of providing a graphical user interface by which a third party accesses the matched sequence database. In a specific embodiment, the business method may further consist of charging a fee to the third party for providing the database. In a further embodiment, the fee may be charged as a one-time fee, a per-consumer fee, or a time-based fee.

In a specific embodiment, a business method may consist of providing a method to produce the matched sequence database. In a specific embodiment, the business method may further consist of charging a fee to a third party using the method. In a further embodiment, the fee may be charged as a one-time fee, a per-consumer fee, or a time-based fee.

In a specific embodiment of the present invention, a microarray may be produced comprising one or more sequences or portions thereof, of the biomolecular sequences of a matched sequence database.

In a specific embodiment, a group of matched sequences may be selected from the matched sequence database.

In a specific embodiment of the present invention, the method comprises creating a matched biomolecular sequence database by comparing information in two or more databases containing biomolecular sequences and selecting entries of biomolecular sequences that are contained in at least two databases. Specifically, a specific embodiment of the methods of the present invention may select entries containing a match between at least a sequence identification in one database and a sequence identification in a second database. Those selected entries may be removed from the two or more databases and placed into the matched biomolecular sequence database. In a specific embodiment, those entries containing a match between a biomolecular sequence and a portion of a consensus or contig sequence may be selected. A specific embodiment may then select those entries containing a match between a biomolecular sequence in one database and the biomolecular sequences of a set of clusters in a second database. Those selected entries may be removed from the two or more databases and placed into the matched biomolecular sequence database. Furthermore, a specific embodiment may then select those entries containing a match between a biomolecular sequence in one database and a biomolecular sequence in a second database within a specified homology. Each selected entry may then be stored in a database.

In an embodiment of the present invention, the database created by performing the above method is described. In a further embodiment of the present invention, the database created by performing the above method on a periodic basis is described.

In an embodiment of the present invention, business methods for using, selling, or distributing the information contained in the database created by performing the methods of the present invention are described. Moreover, business methods for selling or distributing the database created by performing the above method are described.

### BRIEF DESCRIPTION OF THE DRAWINGS

FIG. 1 is a block diagram of a system comprising an embodiment of the present invention.

FIG. 2 is a flow chart of the steps performed by an embodiment of the present invention to update information in a database.

FIG. 3A is a flow chart of the steps performed by an embodiment of the present invention to parse the contents of a flat file database.

FIG. 3B is a flow chart of the steps performed by an embodiment of the present invention to parse the contents of a relational database.

FIG. 4 is a flow chart of the steps performed by an embodiment of the present invention to compare an entry from one database to an entry from another database according to an embodiment of the present invention.

FIG. 5 is a flow chart of the steps performed by an embodiment of the present invention to compile databases of information selected by an embodiment of the present invention.

FIG. 6 is a graphical user interface used to perform a biomolecular sequence information search according to an embodiment of the present invention.

FIG. 7 is a graphical user interface used to perform a biomolecular sequence information search according to an embodiment of the present invention.

FIG. 8 is a graphical user interface containing the result of a biomolecular sequence information search according to an embodiment of the present invention.

### DETAILED DESCRIPTION OF THE INVENTION

It is to be understood that the terminology used herein is for the purpose of describing particular embodiments only, and is not intended to limit the scope of the present invention which will be limited only by the appended claims.

It must be noted that as used herein and in the appended claims, the singular forms “a,” “an,” and “the” include plural references unless the context clearly dictates otherwise. Thus, for example, reference to “a gene” is a reference to one or more genes and includes equivalents thereof known to those skilled in the art, and so forth.

Unless defined otherwise, all technical and scientific terms used herein have the same meaning as commonly understood to one of ordinary skill in the art to which this invention belongs. Although any methods, devices, and materials similar or equivalent to those described herein can be used in the practice or testing of the invention, the preferred methods, devices and materials are now described.

All publications and patents mentioned herein are hereby incorporated herein by reference for the purpose of describing and disclosing, for example, the methodologies that are described in the publications which might be used in connection with the present invention. Publications discussed throughout the text are provided solely for their disclosure prior to the filing date of the present application. Nothing herein is to be construed as an admission that the inventors are not entitled to antedate such disclosure by virtue of prior invention.

Definitions

For convenience, the meaning of certain terms and phrases employed in the specification, examples, and appended claims are provided below. The definitions are not meant to be limiting in nature and serve to provide a clearer understanding of certain aspects of the present invention.

The term “Sequence ID” refers to an alphanumeric identification used to describe an entry in a database, specifically a biomolecular sequence.

The term “Source ID” refers to an alphanumeric identification used to describe the database in which a Sequence ID is found.

The term “gene” refers to a nucleic acid sequence that comprises control and coding sequences necessary for the production of a polypeptide or precursor. The polypeptide can be encoded by a full length coding sequence or by any portion of the coding sequence. The gene may be derived in whole or in part from any source known to the art, including a plant, a fungus, an animal, a bacterial genome or episome, eukaryotic, nuclear or plasmid DNA, CDNA, viral DNA, or chemically synthesized DNA. A gene may contain one or more modifications in either the coding or the untranslated regions that could affect the biological activity or the chemical structure of the expression product, the rate of expression, or the manner of expression control. Such modifications include, but are not limited to, mutations, insertions, deletions, and substitutions of one or more nucleotides. The gene may constitute an uninterrupted coding sequence or it may include one or more introns, bound by the appropriate splice junctions. A biomolecular sequence may comprise all or a portion of a gene.

The term “gene expression” refers to the process by which a nucleic acid sequence undergoes successful transcription and translation such that detectable levels of the nucleotide sequence are expressed.

The term “genome” is intended to include the entire DNA complement of an organism, including the nuclear DNA component, chromosomal or extrachromosomal DNA, as well as the cytoplasmic domain (e.g., mitochondrial DNA).

The term “cell type” refers to a cell from a given source (e.g., tissue, organ) or a cell in a given state of differentiation, or a cell associated with a given pathology or genetic makeup.

The term “microarray” refers to the type of genes or proteins represented on an microarray by oligonucleotides or protein-capture agents, and where the type of genes or proteins represented on the microarray is dependent on the intended purpose of the microarray (e.g., to monitor expression of human genes or proteins). The oligonucleotides or protein-capture agents on a given microarray may correspond to the same type, category, or group of genes or proteins. Genes or proteins may be considered to be of the same type if they share some common characteristics such as species of origin (e.g., human, mouse, rat); disease state (e.g., cancer); functions (e.g., protein kinases, tumor suppressors); same biological process (e.g., apoptosis, signal transduction, cell cycle regulation, proliferation, differentiation). For example, one microarray type may be a “cancer microarray” in which each of the microarray oligonucleotides or protein-capture agents correspond to a gene or protein associated with a cancer. An “epithelial microarray” may be a microarray of oligonucleotides or protein-capture agents corresponding to unique epithelial genes or proteins. Similarly, a “cell cycle microarray” may be a microarray type in which the oligonucleotides or protein-capture agents correspond to unique genes or proteins associated with the cell cycle.

As used herein, the term “support” refers to material having a rigid or semi-rigid surface. Such materials may take the form of plates or slides, small beads, pellets, disks, gels or other convenient forms, although other forms may be used. In some embodiments, at least one surface of the support will be substantially flat. In other embodiments, a roughly spherical shape may be preferred. In the microarrays of the present invention, the oligonucleotide probes or protein-capture agents (defined below) may be directly or indirectly attached or stably associated with a surface of a rigid support, i.e., the probes maintain their position relative to the rigid support under hybridization and washing conditions. As such, the oligonucleotide probes or protein-capture agents may be non-covalently or covalently associated with the support surface. Examples of non-covalent association include non-specific adsorption, specific binding through a specific binding pair member covalently attached to the support surface, and entrapment in a support material (e.g., a hydrated or dried separation medium) which presents the oligonucleotide probe or protein-capture agent in a manner sufficient for hybridization to occur. Examples of covalent binding include covalent bonds formed between the oligonucleotide probe or protein-capture agent and a functional group present on the surface of the rigid support (e.g., —OH) where the functional group may be naturally occurring or present as a member of an introduced linking group.

As mentioned above, the microarray may be present on a rigid support. By rigid, the support is solid and preferably does not readily bend. As such, the rigid supports of the microarrays are sufficient to provide physical support and structure to the oligonucleotide probes or protein-capture agents present thereon under the assay conditions in which the microarray is utilized, particularly under high-throughput handling conditions.

As used herein, the term “protein-capture agent” refers to a molecule or a multi-molecular complex that can bind a protein to itself. In one embodiment, protein-capture agents bind their binding partners in a substantially specific manner. In one embodiment, protein-capture agents may exhibit a dissociation constant (K_{D}) of less than about 10⁻⁶. The protein-capture agent may comprise a biomolecule such as a protein or a polynucleotide. The biomolecule may further comprise a naturally occurring, recombinant, or synthetic biomolecule. Examples of protein-capture agents include antibodies, antigens, receptors, or other proteins, or portions or fragments thereof. Furthermore, protein-capture agents are understood not to be limited to agents that only interact with their binding partners through noncovalent interactions. Rather, protein-capture agents may also become covalently attached to the proteins with which they bind. For example, the protein-capture agent may be photocrosslinked to its binding partner following binding.

The term “spatially directed oligonucleotide synthesis” refers to any method of directing the synthesis of an oligonucleotide to a specific location on a support.

The term “activation” as used herein refers to any alteration of a signaling pathway or biological response including, for example, increases above basal levels, restoration to basal levels from an inhibited state, and stimulation of the pathway above basal levels.

The term “differential expression” refers to both quantitative as well as qualitative differences in the temporal and tissue expression patterns of a gene. For example, a differentially expressed gene may have its expression activated or completely inactivated in normal versus disease conditions. Such a qualitatively regulated gene may exhibit an expression pattern within a given tissue or cell type that is detectable in either control or disease conditions, but is not detectable in both.

The term “cluster” refers to a group of clones or biomolecular sequences related to one another by sequence homology. In one example, clusters are formed based upon a specified degree of homology and/or overlap (e.g., stringency). “Clustering” may be performed with the sequence data. For instance, a biomolecular sequence thought to be associated with a particular molecular or biological function in one tissue might be compared against another library or database of sequences. This type of search is useful to look for homologous, and presumably functionally related, sequences in other tissues or samples, and may be used to streamline the methods of the present invention in that clustering may be used within one or more of the databases to cluster biomolecular sequences prior to performing a method of the invention. The sequences showing sufficient homology with the representative sequence are considered part of a “cluster.” Such “sufficient” homology may vary within the needs of one skilled in the art.

The term “biological sample” refers to a sample obtained from an organism (e.g., patient) or from components (e.g., cells) of an organism. The sample may be of any biological tissue or fluid. The sample may be a “clinical sample” which is a sample derived from a patient. Such samples include, but are not limited to, sputum, blood, blood cells (e.g., white cells), amniotic fluid, plasma, semen, bone marrow, tissue or fine needle biopsy samples, urine, peritoneal fluid, and pleural fluid, or cells therefrom. Biological samples may also include sections of tissues such as frozen sections taken for histological purposes.

The term “amino acid sequence” as used herein includes an oligopeptide, peptide, polypeptide, or protein sequence, and fragment thereof, naturally occurring or synthetic molecules. Biomolecular sequences may comprise amino acid sequences.

The term “sequence database” refers to a database designed to include sequences of biomolecules.

The term “matched sequence database” refers to a database designed to include separate parts, one of which may be a database containing annotation information about sequences in one or more sequence databases, specifically matched sequences. Such information may include, for example, the database (commercial or proprietary) or library in which a given sequence was found, descriptive information about related cDNA associated with the sequence, cellular location, biological and molecular function, cellular pathway, biological process, mapping data, and gene family.

The term “internal database” refers to a database maintained within a local computer network. It contains biomolecular sequences associated with a project. It may also contain information associated with sequences including, but not limited to, a library in which a given sequence is found and descriptive information about a likely gene associated with the sequence. The internal database may typically be maintained as a private database behind a firewall within an enterprise network. However, the invention is not limited to only this embodiment and an internal database could be made available to the public. The internal database may include sequence data generated by the same enterprise that maintains the database, and may also include sequence data obtained from external sources.

The term “external database” refers to a database located outside all internal databases. Typically, an enterprise network differing from the enterprise network maintaining the internal database will maintain an external database. The external database may be used, for example, to provide some descriptive information on biomolecular sequences stored in the internal database. In a specific embodiment, the external database is GenBank and associated databases maintained by the National Center for Biotechnology Information (NCBI), part of the National Library of Medicine.

The term “module” as used herein, refers to a separate unit of computer software or hardware, such as a logical segment of a computer program. A module may be implemented by, for example, a single subroutine or may involve multiple subroutines, or a portion of a single or multiple subroutines. Indeed, in certain embodiments, a module may be implemented entirely by hardware, according to techniques known in the art. The term “module” herein also includes “objects” as the term is used in object oriented programming, as well as equivalent, similar, and analogous programming structures and hardware implementations.

The term “biomolecule” includes nucleic acids and proteins.

The term “biological function” refers to the biological behavior and effects of a protein or peptide. Generally, a protein's biological function does not directly specify its structure or functioning at a molecular level. Rather, it specifies the protein's behavior at least at the cellular level. Examples include “cell signaling” and “DNA repair.”

The term “molecular function” refers to the local or chemical behavior of a protein or peptide. Generally, a protein's molecular function does not account for its functioning at biological level. Examples of molecular function include “receptor” and “calcium channels.”

“BLAST” (Basic Local Alignment Search Tool) is a technique for detecting ungapped sub-sequences that match a given query sequence. BLAST is used in one embodiment of the present invention as a final step in detecting sequence matches.

“BLASTP” is a BLAST program that compares an amino acid query sequence against a protein sequence database.

“BLASTX” is a BLAST program that compares the six-frame conceptual translation products of a nucleotide query sequence (both strands) against a protein sequence database.

The abbreviation “cds” in a GenBank DNA sequence entry refers to the coding sequence. A coding sequence is a sub-sequence of a DNA sequence that is surmised to encode a gene.

A “consensus” or “contig” sequence is a group of assembled overlapping sequences, particularly between sequences in one or more of the databases of the present invention.

Reference will now be made in detail to implementations of the present invention as illustrated in the accompanying drawings. Wherever possible, the same reference numbers will be used throughout the drawings and the following description to refer to the same or like parts.

The present invention embodies systems and methods of comparing and matching information from each of a plurality of databases with similar information in a specific database. Specifically, a specific embodiment of the present invention compares and matches information including, but not limited to, DNA sequences, amino acid sequences, gene sequences, nucleotide sequences, accession numbers, gene mappings, molecular functions, biological processes, and other information relating to DNA, genes, polypeptides, and nucleotides.

Bioinformatics uses computer and statistical techniques to analyze nucleic acid sequence information, and to predict protein sequence, structure and function from DNA sequence data. The present invention relates to biomolecular sequence databases for storing and retrieving biological information. Specifically, the invention relates to methods for providing biomolecular sequences in a format allowing retrieval in a client-server environment.

The methods of the present invention provide biomolecular sequence comparisons and matchings to explore the relationships, for example, between sequence and phenotype. In particular, these resultant matched sequence databases may be utilized to study gene expression and molecular structure. In addition, the databases may be used to determine the sequence and placement of genes and their relationship to other sequences and genes within the genome or to genes. For example, by linkage mapping, a particular disease may be associated with a chromosome; however, the specific gene may be unknown. Thus, the databases of the present invention may then be used to identify the disease-related gene encoded by a particular chromosome, for example, where the particular chromosome or position on a chromosome is searchable on the database.

In one embodiment of the present invention, the databases, including the matched sequence databases, may include various information on a particular biomolecular sequence. For example, the database may include information relating to the chromosomal location, cellular location, biological and molecular function, gene family, phenotype, cellular pathway, biological process, and mapping information.

In a particular embodiment of the present invention, the databases may contain genetic information for a number of organisms, such as mammals, plants, or bacteria. These databases may be used, for example, to decipher the evolutionary development of various proteins.

Database Creation

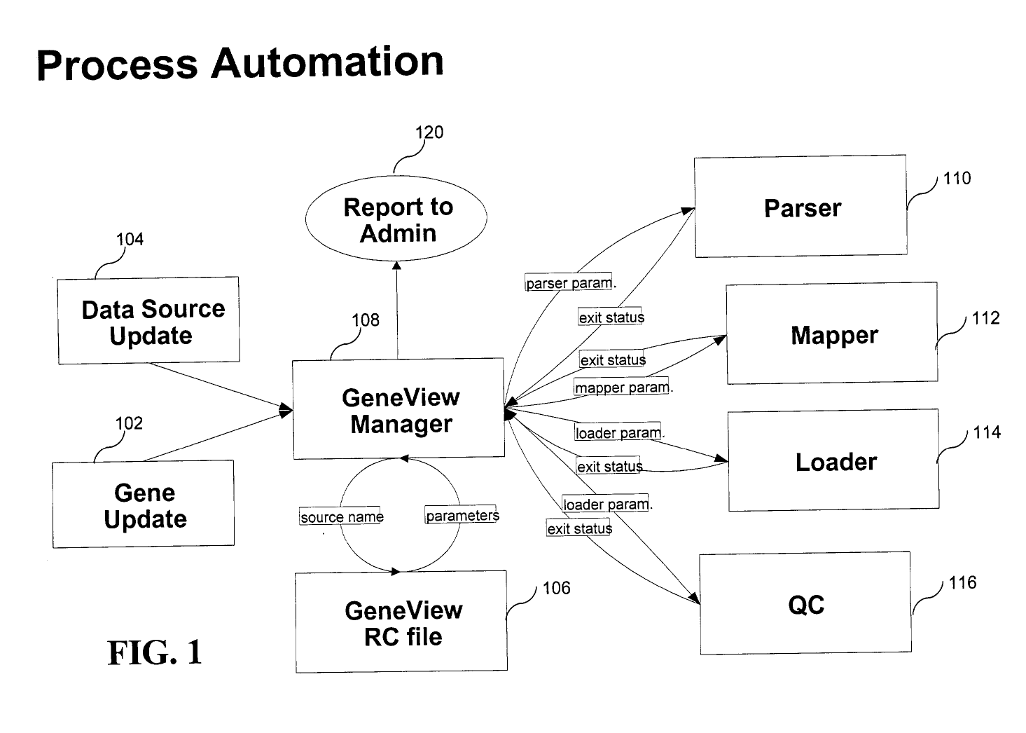

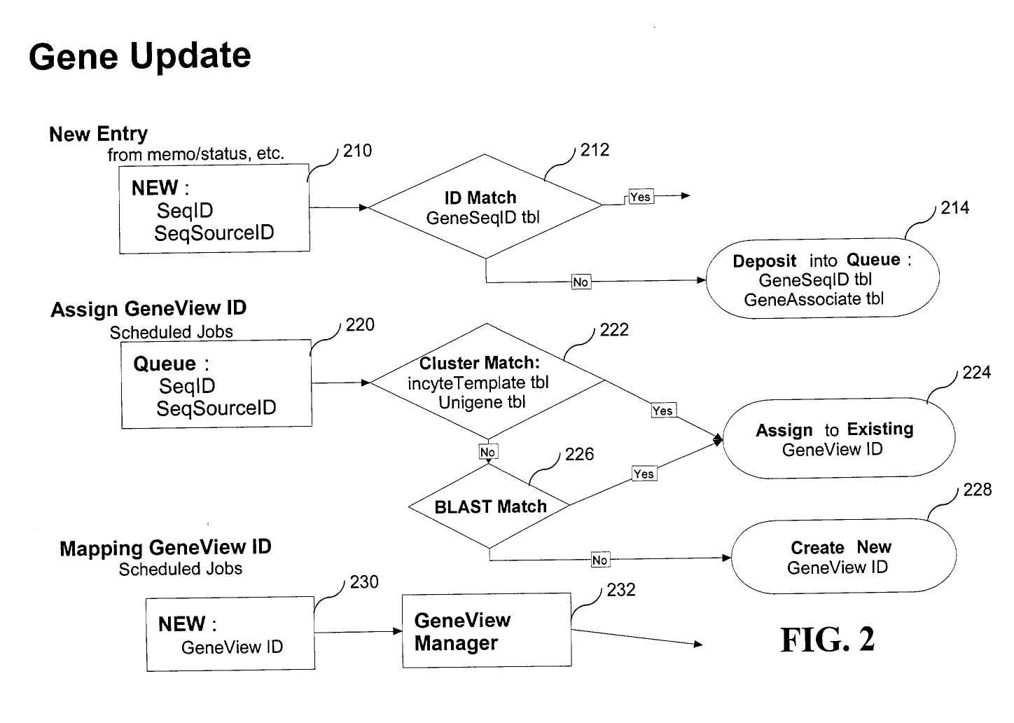

FIG. 1 illustrates an exemplary system that creates a matched sequence database by comparing and matching information from two or more separate databases. First, the specific data sources to be compared are preferably updated. Specifically, a comparison database is updated in the Gene Update process 102, described below in reference to FIG. 2, and an external database is updated in the Data Source Update process 104. Exemplary databases include, but are not limited to, InterPro, Ensembl, dbSNP, OMIM, LocusLink, GeneOntology, UniGene, HomoloGene, Incyte, DNAchip Memo Status, Gene Expression, PRI Classification, and Proteome.

The GeneView RC File 106 contains parameters for each external database. The parameters include, but are not limited to, an Internet, network, World Wide Web, or local computer address of the external database, a timestamp denoting the last known time that the external database was updated, and an Internet, network, World Wide Web, or local computer address denoting where the results of a comparison between the external database and the comparison database are to be stored. In an embodiment of the present invention, the current timestamp denoting the time that an external database was last updated is retrieved from the external database. If the current timestamp differs from the stored timestamp, the current timestamp is stored, and the external database is compared to the comparison database.

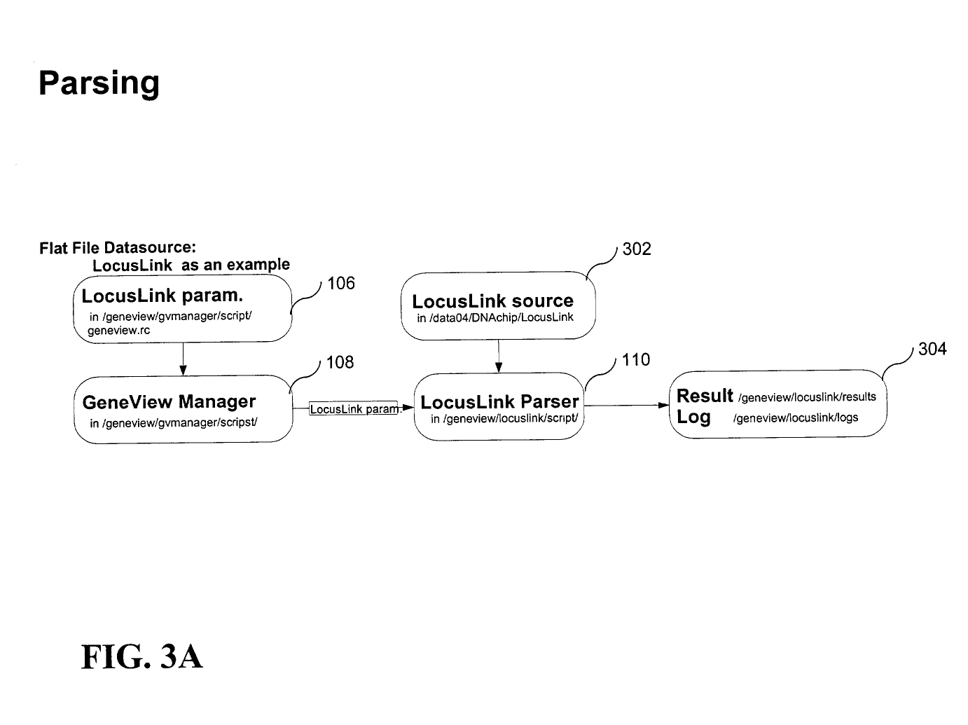

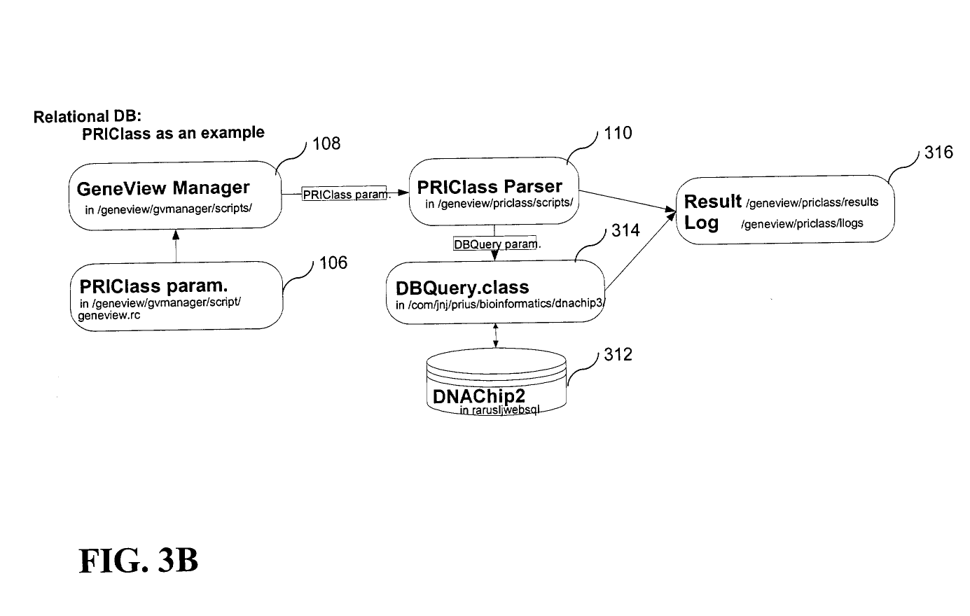

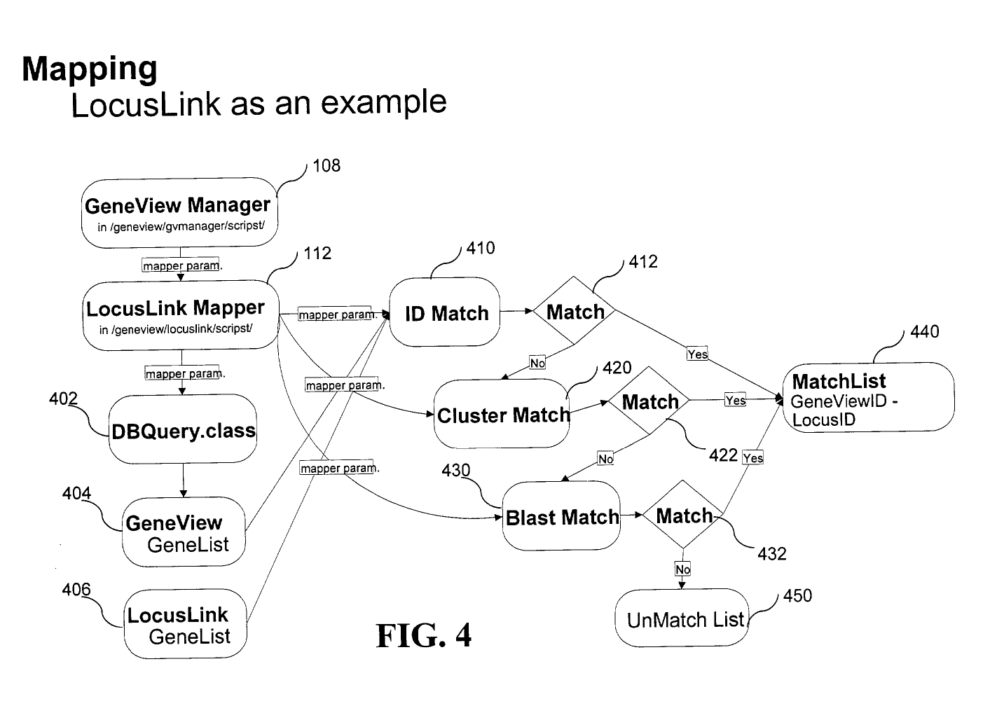

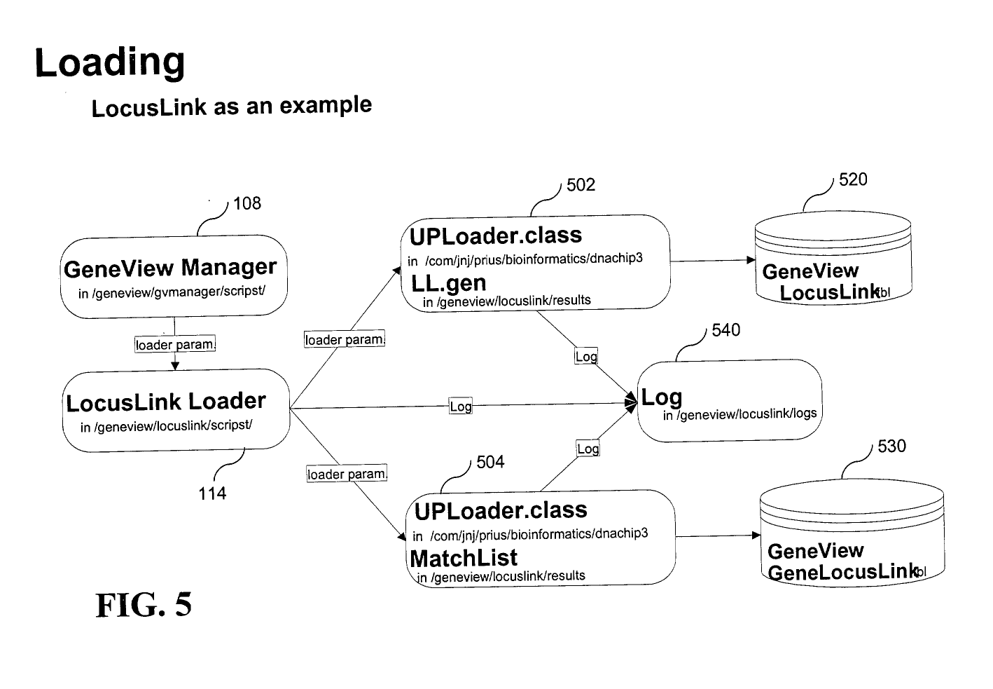

The GeneView Manager 108 acts as a controller and may be implemented in a number of ways including, but not limited to, hardware, software, firmware, and any combination thereof. The GeneView Manager 108 may access parameters stored in the GeneView RC File 106 to determine the location and last update of each database. Moreover, the GeneView Manager 108 may initiate the execution of the Parser 110, as described below in reference to FIGS. 3A and 3B, the Mapper 112, as described below in reference to FIG. 4, and the Loader 114, as described below in reference to FIG. 5. In an embodiment of the present invention, the GeneView Manager 108 may initiate the execution of the QC 116. The GeneView Manager 108 may access the GeneView RC File 106 to retrieve parameters used to assist the execution of the Parser 110, the Mapper 112, the Loader 114, and/or the QC 116. The GeneView Manager 108 may report successful completion or any error conditions to an administrator or an administration module 120.

The Parser 110 may receive parser parameters from the GeneView Manager 108 and may output exit status information. The parser parameters may include, but are not limited to, an Internet, network, World Wide Web, or local computer address of the database to be parsed, the fields of the database to be parsed, and an Internet, network, World Wide Web, or local computer address defining where the output of the Parser 110 is stored. The exit status information may include, but is not limited to, information regarding the successful or unsuccessful completion of the Parser 110 and the output of the Parser 110.

The Mapper 112 may receive mapper parameters from the GeneView Manager 108 and may output exit status information. The mapper parameters may include, but are not limited to, an Internet, network, World Wide Web, or local computer address of the database to be mapped, the fields of the database to be examined by the Mapper 112, and an Internet, network, World Wide Web, or local computer address defining where the output of the Mapper 112 is stored. The exit status information may include, but is not limited to, information regarding the successful or unsuccessful completion of the Mapper 112 and the output of the Mapper 112.

The Loader 114 may receive loader parameters from the GeneView Manager 108 and may output exit status information. The loader parameters may include, but are not limited to, an Internet, network, World Wide Web, or local computer address of the database to be uploaded, and one or more Internet, network, World Wide Web, or local computer addresses defining where the output of the Loader 114 is stored. The exit status information may include, but is not limited to, information regarding the successful or unsuccessful completion of the Loader 114 and the output of the Loader 114.

The QC 116 may receive quality parameters from the GeneView Manager 108 and may output exit status information. The quality parameters may include, but are not limited to, an Internet, network, World Wide Web, or local computer address of the database to be uploaded, the fields of the database to examine when performing quality control operations, and one or more Internet, network, World Wide Web, or local computer addresses defining where the output of the QC 116 is stored. The exit status information may include, but is not limited to, information regarding the successful or unsuccessful completion of the QC 116 and the output of the QC 116.

FIG. 2 illustrates the operation of the Gene Update process 102 for the comparison database. The operation of the Gene Update process 102 may include, but is not limited to, adding new entries 210, assigning GeneView IDs 220, and mapping GeneView IDs 230.

When a new entry is added to the comparison database 210, the Gene Update process 102 may compare the Sequence ID and Source ID of the new entry to entries in a Biomolecular Sequence ID table of the comparison database, as in 212. If there is a direct match between the Sequence ID and Source ID of the new entry, and an entry in the Biomolecular Sequence ID table of the comparison database, the new entry may be discarded. If there is not a direct match between the Sequence ID and Source ID of the new entry, and any entry in the Biomolecular Sequence ID table of the comparison database, the new entry may be added to the Biomolecular Sequence ID table and a Gene Associate table 214. Moreover, the Sequence ID and Source ID of the new entry may be placed on a queue 214 to determine the GeneView ID to assign to the new entry.

When assigning a GeneView ID 220, the Gene Update process 102 removes the Sequence ID and Source ID for an entry from the queue. The process may then perform a Cluster Match 222 on the Sequence ID and Source ID based on one or more cluster tables. The cluster tables may include, but are not limited to, an Incyte table and a Unigene table. If the Cluster Match 222 returns a match between the new entry and a known cluster, the GeneView ID of the cluster may be assigned to the new entry 224. If no match is found, a BLAST Match 226 may be performed on the new entry against all entries in the comparison database. If the BLAST Match 226 returns a match between the new entry and a known biomolecular sequence in the comparison database, the GeneView ID of the known biomolecular sequence may be assigned to the new entry 224. If no match is found, a new GeneView ID may be created for the new entry 228.

When a new GeneView ID has been assigned to a new entry 230, the Gene Update process 102 may inform the GeneView Manager 108 of the new entry and map the new GeneView ID against external databases 232.

FIGS. 3A and 3B depict the operation of the Parser 110. The operation of the Parser 110 may depend upon the type of database to be parsed. FIG. 3A depicts the operation of the Parser 110 when the external database is a flat file, such as, for example, LocusLink. FIG. 3B depicts the operation of the Parser 110 when the external database is a relational database, such as, for example, PRI Classification.

In FIG. 3A, when the external database is a flat file, the Parser 110 may load parameters from the GeneView RC File 106 corresponding to the particular external database via the GeneView Manager 108. These parameters may then be used to determine the location of the external database, parse the data retrieved from the external database 302, and store the results in a result file and a log file 304.

In FIG. 3B, when the external database is a relational database, the Parser 110 may load parameters from the GeneView RC File 106 corresponding to the particular external relational database via the GeneView Manager 108. These parameters may then be used to determine the location of the external relational database, generate one or more queries to the external relational database 312, retrieve the results of the one or more queries from the external relational database 314, and store the results in a result file and a log file 316.

FIG. 4 illustrates the operation of the Mapper 112 for mapping the comparison database against a specific external database. In the exemplary embodiment depicted in FIG. 4, the Mapper 112 maps the comparison database 404 against the LocusLink external database 406. However, the Mapper 112 depicted may be used to map the comparison database 404 to other external databases, as will be evident to one of skill in the art.

The Mapper 112 retrieves the mapper parameters from the GeneView RC File 106 via the GeneView Manager 108. The Mapper 112 may use the mapping parameters, inter alia, to perform database queries 402 on an external database 406 and the comparison database 404. The Mapper 112 may perform up to three matching steps on each entry in the comparison database 404 in the following order: an ID Match 410, a Cluster Match 420, and a BLAST Match 430. If any of the three matching steps return a positive result on an entry in the comparison database, the remaining matching steps, if any, may not be performed.

The ID Match step 410 compares the Sequence ID and Source ID of an entry in the comparison database 404 with the Sequence ID and Source ID of each entry in the external database 406. If a match is found 412, the entry in the comparison database 404 is added to a Match List 440. If a match is not found 412, a Cluster Match step 420 is performed on the remaining entries in the comparison database 404.

The nucleic acid sequences available in the public databases often represent partial sequences, or expressed sequence tags (ESTs). In one embodiment of the present invention, the databases may be used to compile or cluster overlapping sequences, resulting in the generation of a consensus sequence. For example, a cluster grouping of at least partially overlapping sequences may be aligned using a sequence assembly algorithm. The alignments are influenced by the quality scores assigned to the individual bases of the sequence fragments during the sequencing calling processes. Thus, the result of this alignment process is the assembly of a number of overlapping contiguous DNA sequences into, for example, a full-length gene. Such consensus or contig sequences may be used in the Cluster Match step 420.

The Cluster Match step 420 compares the biomolecular sequence of the entry in the comparison database 404 with each entry in a set of clusters in the external database 406. If a match is found 422, the entry in the comparison database 404 may be added to the Match List 440. If a match is not found 422, a BLAST Match step 430 may be performed on the remaining entries in the comparison database 404.

The BLAST Match step 430 compares the biomolecular sequence of the entry in the comparison database 404 with the biomolecular sequence of each entry in the external database 406. If a match is found 432, the entry in the comparison database 404 is added to the Match List 440. If a match is not found 432, the entry in the comparison database 404 is added to an Unmatched List 450. The entries in the Unmatched List 450 may be excluded from further processing steps.

In an alternate embodiment of the present invention, the ID Match step 410 compares the Sequence ID and Source ID of an entry in the external database 406 with the Sequence ID and Source ID of each entry in the comparison database 404. If a match is found 412, the entry in the external database 406 is added to a Match List 440. If a match is not found, a Cluster Match step 420 is performed on the remaining entries in the external database 406.

The Cluster Match step 420 compares the biomolecular sequence of the entry in the external database 406 with each entry in a set of clusters in the comparison database 404. If a match is found 422, the entry in the external database 406 is added to the Match List 440. If a match is not found 422, a BLAST Match step 430 is performed on the remaining entries in the external database 406.

The BLAST Match step 430 compares the biomolecular sequence of the entry in the external database 406 with the biomolecular sequence of each entry in the comparison database 404. If a match is found, the entry in the external database 406 may be added to the Match List 440. If a match is not found, the entry in the external database 406 may be added to an Unmatched List 450. The entries in the Unmatched List 450 may be excluded from further processing steps.

FIG. 5 illustrates the operation of the Loader 114 for producing output tables based on the output of the Mapper 112. In the exemplary embodiment depicted in FIG. 5, the Loader 114 produces output tables using the output of the particular Mapper 112 that compares the comparison database 404 with, for example, the LocusLink external database 406. However, a Loader 114 performing substantially similar steps may be used to produce output tables using the output of a Mapper 112 that compares the comparison database 404 with a different external database 406, as will be evident to one of skill in the relevant art.

The Loader 114 retrieves the loader parameters from the GeneView RC File 106 via the GeneView Manager 108. The Loader 114 may use the loader parameters, inter alia, to determine the format and location of the output tables. Specifically, as shown in FIG. 5, the Loader 114 may output a GeneView LocusLink table 520 and a GeneView GeneLocusLink table 530 containing information compiled in the Match List 440. Moreover, a Log file 540 may be created that lists the steps taken in the creation of the output tables by the Loader 114.

Database Access

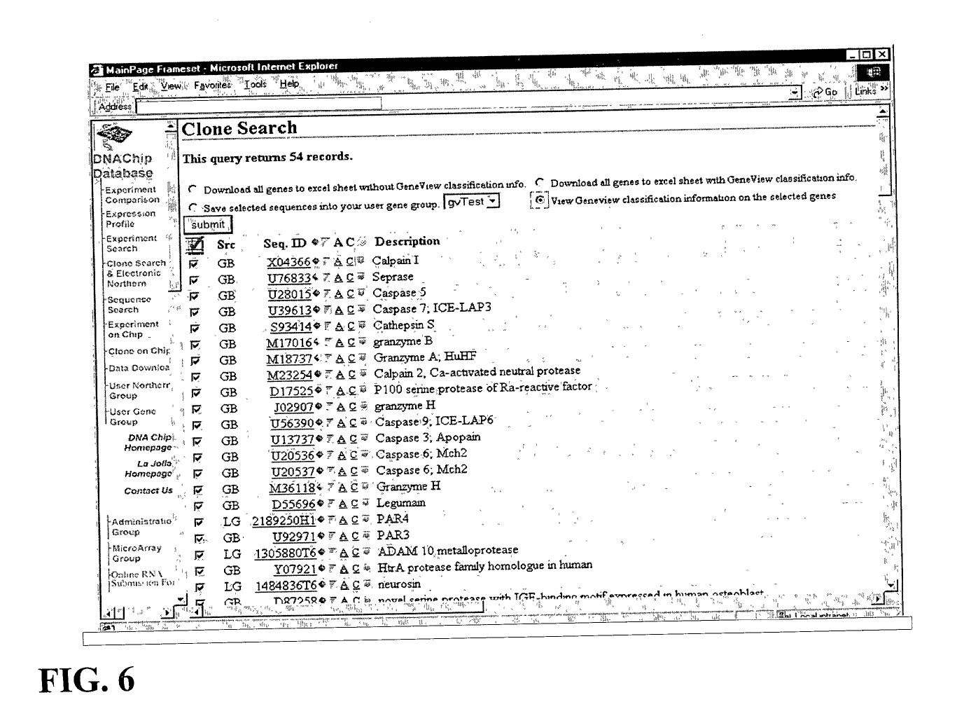

FIG. 6 illustrates an example Graphical User Interface (“GUI”) 600 for accessing information stored in a database created by the method described above. In an embodiment, the GUI 600 may be composed of two frames. A first frame may comprise a selectable list of databases accessible by the user. When a database is selected in the first frame, a second frame may display information resulting from the pair-wise comparison of the comparison database with the selected database as described above.

The second frame of the GUI may comprise a listing of biomolecular sequences contained in the selected database. Furthermore, the second frame may allow the user to select a subset, including all of the biomolecular sequences, and to perform an operation on the list of biomolecular sequences. In an embodiment, the user may select the subset of biomolecular sequences by selecting a selection box associated with each biomolecular sequence. In a specific embodiment, the operations that may be performed include, but are not limited to, downloading all listed biomolecular sequences to a database spreadsheet with classification information, saving the selected subset of biomolecular sequences to a user file, downloading all listed biomolecular sequences to a database spreadsheet without classification information, and displaying classification information on a selected subset of biomolecular sequences.

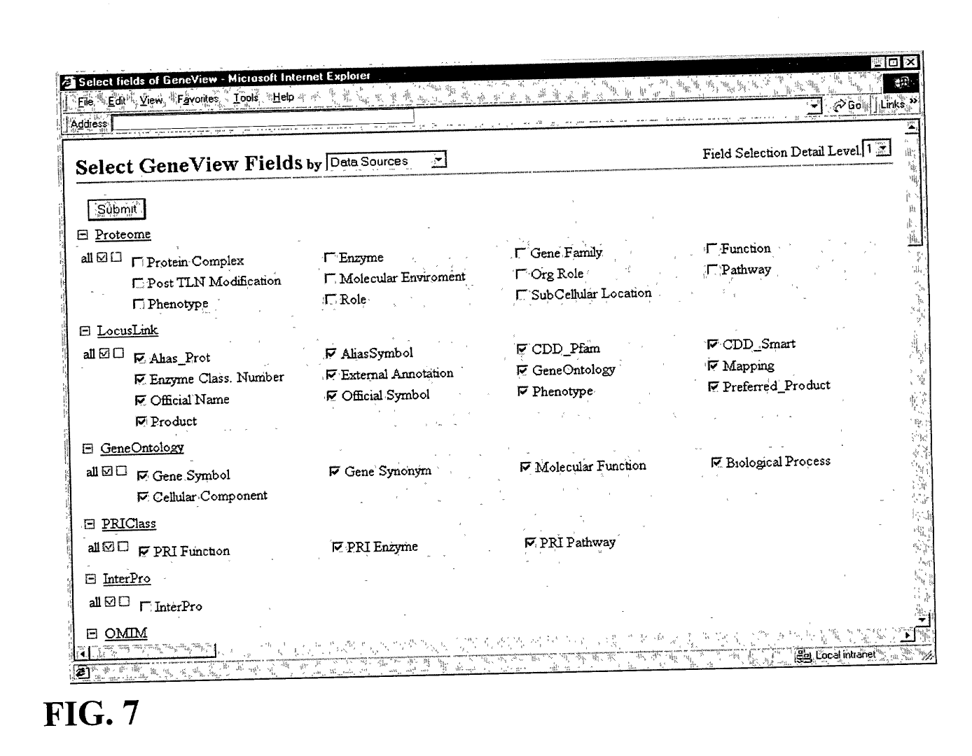

If the user chooses to display classification information on a selected subset of biomolecular sequences, a second GUI may be presented to the user, as illustrated in FIG. 7. In a specific embodiment, the second GUI may contain a listing of one or more external databases used to create matched biomolecular sequence databases as described above. Furthermore, for each external database, the GUI may display a list of one or more fields associated with each external database. In a specific embodiment, the GUI may allow the user to select or deselect each of the one or more fields displayed in the second GUI. In a specific embodiment, the GUI may allow the user to select or deselect each of the one or more external databases.

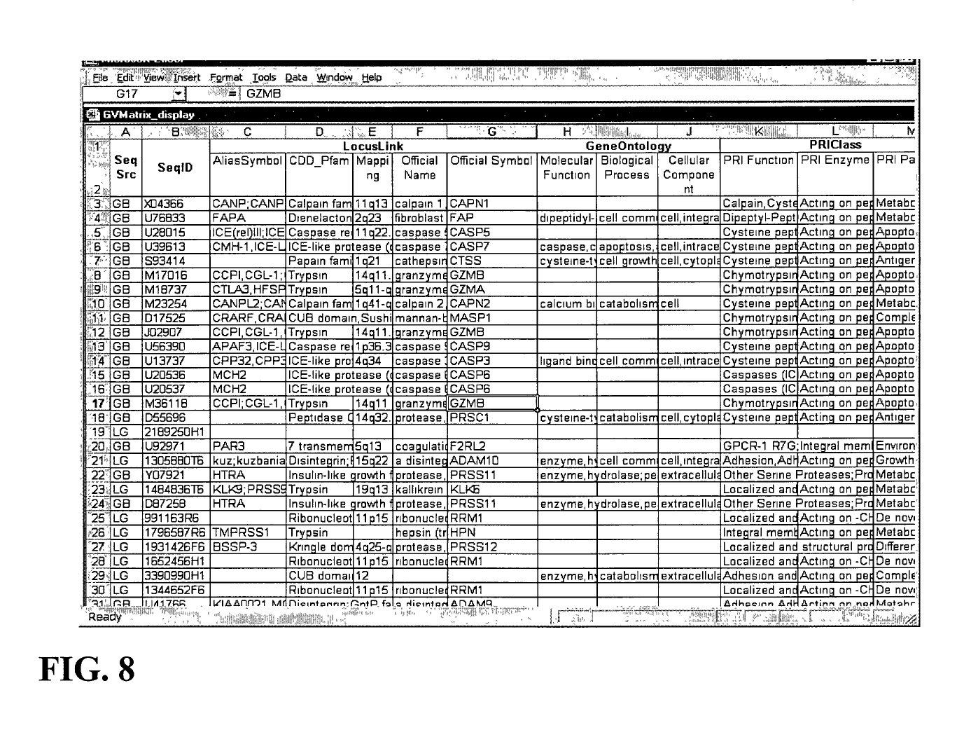

FIG. 8 illustrates an example result of performing a classification information display request. In a specific embodiment, the result of performing a classification information display request may contain a number of fields including, but not limited to, a Source ID, a Sequence ID, and a list of external databases on which the classification information display request was performed. In a specific embodiment, one or more fields may be listed under each external database header representing the classification information requested from the second GUI in FIG. 7. If no information is retrieved from an external database as a result of the classification information display request for a field, the corresponding field in the result may display no data.

An embodiment of the present invention comprises a variety of business methods including methods for providing matched sequence databases to customers, as well as methods for producing matched sequence databases. A further embodiment of the present invention comprises a business method of providing matched sequence databases, and methods for producing such databases, for normal and diseased tissues. Also within the scope of this invention are business methods providing diagnostics and predictors relating to genes and biomolecules.

The business methods of the present application relate to the commercial and other uses of the methodologies of the present invention. In one aspect, the business methods include the marketing, sale, or licensing of the present methodologies in the context of providing consumers, i.e., patients, medical practitioners, medical service providers, researchers, and pharmaceutical distributors and manufacturers, with the matched sequence databases provided by the present invention.

The matched sequence database may be an internal database designed to include annotation information about the matched sequences generated by the methods of the present invention. Such information may include, for example, the databases in which a given nucleic acid sequence was found, descriptive information about related cDNA associated with the sequence, tissue or cell source, sequence data obtained from external sources, and preparation methods. The database may be divided into two sections: one for storing the sequences and the other for storing the associated information. This database may be maintained as a private database with a firewall within the central computer facility. However, this invention is not so limited and the gene expression profile database may be made available to the public.

The database may be a network system connecting the network server with clients. The network may be any one of a number of conventional network systems, including a local area network (LAN) or a wide area network (WAN), as is known in the art (e.g., Ethernet). The server may include software to access database information for processing user requests, and to provide an interface for serving information to client machines. The server may support the World Wide Web and maintain a website and Web browser for client use. Client/server environments, database servers, and networks are well documented in the technical, trade, and patent literature.

Through the Web browser, clients may construct search requests for retrieving data from a microarray database and a gene expression database. For example, the user may “point and click” to user interface elements such as buttons, pull down menus, and scroll bars. The client requests may be transmitted to a Web application that formats them to produce a query that may be used to gather information from the matched sequence database. In addition, the website may provide hypertext links to public databases such as GenBank and associated databases maintained by the National Center for Biotechnology Information (NCBI), part of the National Library of Medicine as well as, any links providing relevant information for gene expression analysis, genetic disorders, scientific literature, and the like.

The present invention also provides a system for accessing and comparing bioinformation, specifically microarray databases, gene expression databases and other information which is useful in the context of the systems and methods of the present invention. In one embodiment, the computer system may comprise a computer processor, suitable memory that is operatively coupled to the computer processor, and a computer process stored in the memory that executes in the computer processor and which comprises a means for matching a gene expression profile of a biomolecular sequence from a patient with expression profile and sequence identification information of biomolecular sequences in a database. More specifically, the computer system is used to match an biomolecular sequence profile generated from a biological sample with a microarray database and/or a gene expression database and other information in a database.

Furthermore, the system for accessing and comparing information contained in biomolecular databases comprises a computer program comprising computer code providing an algorithm for matching an expression profile generated from a patient, with expression profile and sequence identification information of biomolecular sequences in a biomolecular database.

The methods of the present application further relate to the commercial and other uses of the systems and methodologies of the present invention. In one aspect, the methods include the marketing, sale, or licensing of the systems and methodologies of the present invention in the context of providing consumers, i.e., patients, medical practitioners, medical service providers, researchers, and pharmaceutical distributors and manufacturers, with access to biomolecular databases including, in particular, databases produced in accordance with the methodologies and systems of the present invention. One embodiment of the present invention comprises charging users a one-time fee to access the matched biomolecular sequence databases. In a further embodiment, the present invention contemplates charging a time-based fee to the consumer accessing the matched sequence database.

In another embodiment, the methods of the present invention include establishing a distribution system for distributing the methodologies and systems of the present invention for sale, and may optionally include establishing a sales group for marketing the systems and methodologies of the present invention. Yet another aspect of the present invention provides a method of accessing biomolecular sequence information and providing the matched sequence biomolecular sequence information to a consumer and optionally licensing or selling, the rights for access to the matched sequence database.

Methods for Producing Polynucleotide Microarrays

The present invention also relates to the generation of microarrays comprising the biomolecular sequence information generated by the systems and method of the present invention. The microarrays may be produced through spatially directed oligonucleotide synthesis. Methods for spatially directed oligonucleotide synthesis include, without limitation, light-directed oligonucleotide synthesis, microlithography, application by ink jet, microchannel deposition to specific locations and sequestration with physical barriers. In general, these methods involve generating active sites, usually by removing protective groups, and coupling to the active site a nucleotide that, itself, optionally has a protected active site if further nucleotide coupling is desired.

A microarray may be configured, for example, by in situ synthesis or by direct deposition (“spotting” or “printing”) of synthesized oligonucleotide probes onto the support. The oligonucleotide probes are used to detect complementary polynucleotide sequences in a target sample of interest. In situ synthesis has several advantages over direct placement such as higher yields, consistency, efficiency, cost, and potential use of combinatorial strategies (Southern et al. (1999)). However, for longer polynucleotide sequences such as PCR products, deposition may be the preferred method. Generation of microarrays by in situ synthesis may be accomplished by a number of methods including photochemical deprotection, ink-jet delivery, and flooding channels (Lipshutz et al., 21 NATURE GENET. 20-24 (1999); Blanchard et al., 11 BIOSENSORS AND BIOELECTRONICS, 687-90 (1996); Maskos et al., 21 NUCLEIC ACIDS RES. 4663-69 (1993)).

The present invention relates to the construction of microarrays by the in situ synthesis method using solid-phase DNA synthesis and photolithography (Lipshutz et al. (1999)). Linkers with photolabile protecting groups may be covalently or non-covalently attached to a support (e.g., glass). Light is then directed through a photolithographic screen to specific areas on the support resulting in localized photodeprotection and yielding reactive hydroxyl groups in the illuminated regions. A 3′-O-phosphoramidite-activated deoxynucleoside (protected at the 5′-hydroxyl with a photolabile group) is then incubated with the support and coupling occurs at deprotected sites that were exposed to light. Following the optional capping of unreacted active sites and oxidation, the support is rinsed and the surface is illuminated through a second screen, to expose additional hydroxyl groups for coupling to the linker. A second 5′-protected, 3′-O-phosphoramidite-activated deoxynucleoside is presented to the support. The selective photodeprotection and coupling cycles are repeated until the desired products are obtained. Photolabile groups may then be removed and the sequence may be capped. Side chain protective groups may also be removed. Because photolithography is used, the process may be miniaturized to generate high-density microarrays of oligonucleotide probes. Thus, thousands to hundreds of thousands of arbitrary oligonucleotide probes may be generated on a single microarray support using this technology.

To produce a microarray by the spotting method, oligonucleotide probes are prepared, generally by PCR, for printing onto the microarray support. As described for the in situ technique, the probes may be selected from a number of sources including polynucleotide databases such as GenBank, Unigen, HomoloGene, RefSeq, dbEST, and dbSNP (Wheeler et al., 29 NUCLEIC ACIDS RES. 11-16 (2001)). In addition, oligonucleotide probes may be randomly selected from cDNA libraries reflecting, for example, a tissue type (e.g., cardiac or neuronal tissue), or a genomic library representing a species of interest (e.g., Drosophilia melanogaster). If PCR is used to generate the probes, for example, approximately 100-500 pg of the purified PCR product (about 0.6-2.4 kb) may be spotted onto the support (Duggan et al., 21 NATURE GENET. 10-14 (1999)). The spotting (or printing) may be performed by a robotic arrayer (see, e.g., U.S. Pat. Nos. 6,150,147; 5,968,740; 5,856,101; 5,474,796; and 5,445,934;).

A number of different microarray configurations and methods for their production are known to those of skill in the art and are disclosed in U.S. Pat. Nos.: 6,156,501; 6,077,674; 6,022,963; 5,919,523; 5,885,837; 5,874,219; 5,856,101; 5,837,832; 5,770,722; 5,770,456; 5,744,305; 5,700,637; 5,624,711; 5,593,839; 5,571,639; 5,556,752; 5,561,071; 5,554,501; 5,545,531; 5,529,756; 5,527,681; 5,472,672; 5,445,934; 5,436,327; 5,429,807; 5,424,186; 5,412,087; 5,405,783; 5,384,261; 5,242,974; and the disclosures of which are herein incorporated by reference. Patents describing methods of using arrays in various applications include: U.S. Pat. Nos. 5,874,219; 5,848,659; 5,661,028; 5,580,732; 5,547,839; 5,525,464; 5,510,270; 5,503,980; 5,492,806; 5,470,710; 5,432,049; 5,324,633; 5,288,644; 5,143,854; and the disclosures of which are incorporated herein by reference.

Microarray Supports

A microarray support may comprise a flexible or rigid support. A flexible support is capable of being bent, folded, or similarly manipulated without breakage. Examples of solid materials that are flexible solid supports with respect to the present invention include membranes, such as nylon and flexible plastic films. The rigid supports of microarrays are sufficient to provide physical support and structure to the associated oligonucleotides under the appropriate assay conditions.

The support may be biological, nonbiological, organic, inorganic, or a combination of any of these, existing as particles, strands, precipitates, gels, sheets, tubing, spheres, containers, capillaries, pads, slices, films, plates, or slides. In addition, the support may have any convenient shape, such as a disc, square, sphere, or circle. In one embodiment, the support is flat but may take on a variety of alternative surface configurations. For example, the support may contain raised or depressed regions on which the synthesis takes place. The support and its surface may form a rigid support on which the reactions described herein may be carried out. The support and its surface may also be chosen to provide appropriate light-absorbing characteristics. For example, the support may be a polymerized Langmuir Blodgett film, functionalized glass, Si, Ge, GaAs, GaP, Sio₂, SIN₄, modified silicon, or any one of a wide variety of gels or polymers such as (poly)tetrafluoroethylene, (poly)vinylidenedifluoride, polystyrene, polycarbonate, or combinations thereof. The surface of the support may also contain reactive groups, such as carboxyl, amino, hydroxyl, and thiol groups. The surface may be transparent and contain SiOH functional groups, such as found on silica surfaces.

The support may be composed of a number of materials including glass. There are several advantages for utilizing glass supports in constructing a microarray. For example, microarrays prepared using a glass support, generally utilize microscope slides due to the low inherent fluorescence, thus, minimizing background noise. Moreover, hundreds to thousands of oligonucleotide probes may be attached to slide. The glass slides may be coated with polylysine, amino silanes, or amino-reactive silanes that enhance the hydrophobicity of the slide and improve the adherence of the oligonucleotides (Duggan et al., 21 NATURE GENET. 10-14 (1999)). Ultraviolet irradiation is used to crosslink the oligonucleotide probes to the glass support. Following irradiation, the support may be treated with succinic anhydride to reduce the positive charge of the amines. For double-stranded oligonucleotides, the support may be subjected to heat (e.g., 95° C.) or alkali treatment to generate single-stranded probes. An additional advantage to using glass is its nonporous nature, thus, requiring a minimal volume of hybridization buffer resulting in enhanced binding of target samples to probes.

In another embodiment, the support may be flat glass or single-crystal silicon with surface relief features of less than about 10 angstroms. The surface of the support may be etched using well-known techniques to provide desired surface features. For example, trenches, v-grooves, or mesa structures allow the synthesis regions to be more closely placed within the focus point of impinging light.

The present invention also contemplates polynucleotide microarray supports comprising beads. These beads may have a wide variety of shapes and may be composed of numerous materials. Generally, the beads used as supports may have a homogenous size between about 1 and about 100 microns, and may include microparticles made of controlled pore glass (CPG), highly crosslinked polystyrene, acrylic copolymers, cellulose, nylon, dextran, latex, and polyacrolein. See e.g., U.S. Pat. Nos. 6,060,240; 4,678,814; and 4,413,070.

Several factors may be considered when selecting a bead for a support including material, porosity, size, shape, and linking moiety. Other important factors to be considered in selecting the appropriate support include uniformity, efficiency as a synthesis support, surface area, and optical properties (e.g., autofluoresence). Typically, a population of uniform oligonucleotide or polynucleotide fragment may be employed. However, beads with spatially discrete regions each containing a uniform population of the same oligonucleotide or polynucleotide fragment (and no other), may also be employed. In one embodiment, such regions are spatially discrete so that signals generated by fluorescent emissions at adjacent regions can be resolved by the detection system being employed.

In general, the support beads may be composed of glass (silica), plastic (synthetic organic polymer), or carbohydrate (sugar polymer). A variety of materials and shapes may be used, including beads, pellets, disks, capillaries, cellulose beads, pore-glass beads, silica gels, polystyrene beads optionally crosslinked with divinylbenzene, grafted co-poly beads, polyacrylamide beads, latex beads, dimethylacrylamide beads optionally cross-linked with N,N-1-bis-acryloyl ethylene diamine, and glass particles coated with a hydrophobic polymer (e.g., a material having a rigid or semirigid surface). The beads may also be chemically derivatized so that they support the initial attachment and extension of nucleotides on their surface.

Oligonucleotide probes, including probes specific for GPCR polynucleotides, may be synthesized directly on the bead, or the probes may be separately synthesized and attached to the bead. See, e.g., Albretsen et al., 189 ANAL. BIOCHEM. 40-50 (1990); Lund et al., 16 NUCLEIC ACIDS RES. 10861-80 (1988); Ghosh et al., 15 NUCLEIC ACIDS RES. 5353-72 (1987); Wolf et al., 15 NUCLEIC ACIDS RES. 2911-26 (1987). The attachment to the bead may be permanent, or a cleavable linker between the bead and the probe may also be used. The link should not interfere with the probe-target binding during screening. Linking moieties for attaching and synthesizing tags on microparticle surfaces are disclosed in U.S. Pat. No. 4,569,774; Beattie et al., 39 CLIN. CHEM. 719-22 (1993); Maskos and Southern, 20 NUCLEIC ACIDS RES. 1679-84 (1992); Damba et al., 18 NUCLEIC ACIDS RES. 3813-21 (1990); and Pon et al., 6 BIOTECHNIQUES 768-75 (1988). Various links may include polyethyleneoxy, saccharide, polyol, esters, amides, saturated or unsaturated alkyl, aryl, and combinations thereof.

If the oligonucleotide probes are chemically synthesized on the bead, the bead-oligo linkage may be stable during the deprotection step of photolithography. During standard phosphoramidite chemical synthesis of oligonucleotides, a succinyl ester linkage may be used to bridge the 3′ nucleotide to the resin. This linkage may be readily hydrolyzed by NH₃ prior to and during deprotection of the bases. The finished oligonucleotides may be released from the resin in the process of deprotection. The probes may be linked to the beads by a siloxane linkage to Si atoms on the surface of glass beads; a phosphodiester linkage to the phosphate of the 3′-terminal nucleotide via nucleophilic attack by a hydroxyl (typically an alcohol) on the bead surface; or a phosphoramidate linkage between the 3′-terminal nucleotide and a primary amine conjugated to the bead surface.

Numerous functional groups and reactants may be used to detach the oligonucleotide probes. For example, functional groups present on the bead may include hydroxy, carboxy, iminohalide, amino, thio, active halogen (Cl or Br) or pseudohalogen (e.g., CF₃, CN), carbonyl, silyl, tosyl, mesylates, brosylates, and triflates. In some instances, the bead may have protected functional groups that may be partially or wholly deprotected.

Microarray Support Surface

The support of the microarrays may comprise at least one surface on which a pattern of biomolecular seqeunces is present, where the surface may be smooth or substantially planar, or have irregularities, such as depressions or elevations. The surface on which the probes are located may be modified with one or more different layers of compounds that serve to modulate the properties of the surface. Such modification layers may generally range in thickness from a monomolecular thickness of about 1 mm, preferably from a monomolecular thickness of about 0.1 mm, and most preferred from a monomolecular thickness of about 0.001 mm. Modification layers include, for example, inorganic and organic layers such as metals, metal oxides, polymers, small organic molecules and the like. Polymeric layers include peptides, proteins, polynucleotides or mimetics thereof (e.g., peptide nucleic acids), polysaccharides, phospholipids, polyurethanes, polyesters, polycarbonates, polyureas, polyamides, polyethyleneamines, polyarylene sulfides, polysiloxanes, polyimides, and polyacetates. The polymers may be hetero- or homopolymeric, and may or may not have separate functional moieties attached.

The oligonucleotide probes of a microarray may be arranged on the surface of the support based on size. With respect to the arrangement according to size, the probes may be arranged in a continuous or discontinuous size format. In a continuous size format, each successive position in the microarray, for example, a successive position in a lane of probes, comprises oligonucleotide probes of the same molecular weight. In a discontinuous size format, each position in the pattern (e.g., band in a lane) represents a fraction of target molecules derived from the original source, where the probes in each fraction will have a molecular weight within a determined range.

The probe pattern may take on a variety of configurations as long as each position in the microarray represents a unique size (e.g., molecular weight or range of molecular weights), depending on whether the microarray has a continuous or discontinuous format. The microarrays may comprise a single lane or a plurality of lanes on the surface of the support. Where a plurality of lanes are present, the number of lanes will usually be at least about 2 but less than about 200 lanes, preferably more than about 5 but less than about 100 lanes, and most preferred more than about 8 but less than about 80 lanes.

Each microarray may contain oligonucleotide probes isolated from the same source (e.g., the same tissue), or contain probes from different sources (e.g., different tissues, different species, disease and normal tissue). As such, probes isolated from the same source may be represented by one or more lanes; whereas probes from different sources may be represented by individual patterns on the microarray where probes from the same source are similarly located. Therefore, the surface of the support may represent a plurality of patterns of oligonucleotide probes derived from different sources (e.g., tissues), where the probes in each lane are arranged according to size, either continuously or discontinuously.

Surfaces of the support are usually, though not always, composed of the same material as the support. Alternatively, the surface may be composed of any of a wide variety of materials, for example, polymers, plastics, resins, polysaccharides, silica or silica-based materials, carbon, metals, inorganic glasses, membranes, or any of the above-listed support materials. The surface may contain reactive groups, such as carboxyl, amino, or hydroxyl groups. The surface may be optically transparent and may have surface SiOH functionalities, such as are found on silica surfaces.

### EXAMPLES

The present invention is further illustrated by the following examples, which should not be construed as limiting in any way. The contents of all cited references (including literature references, issued patents, published patent applications, and co-pending patent applications) cited throughout this application are hereby expressly incorporated by reference.

### Example 1

Algorithm to Match Gene Sets

Different database systems may use different identifiers to describe the same collection of genes. In one embodiment, the present invention develops a composite method, which applies different match and filter algorithms in a specific order. This mapping procedure is more accurate and less computationally intensive when compared with traditional matching based purely on the biomolecular sequence.

Two collections of gene sets, A and B, are constructed. The algorithm of the module creates a one way match from A to B. Each gene entry in set A will be linked to either zero or one gene entry in set B. Information including, but not limited to, identifiers, identifier types, biomolecular sequences, common cluster identifiers (GenBank, Unigene, Incyte template identifiers, etc.) and species names associated with each gene, is retrieved for both set A and set B.

Because multiple entries in a gene set may represent the same gene, an initial sequence clustering is performed on gene set A. Entries that belong to the same cluster are combined into one gene entry. This eliminates the possibility of a many-to-one match from A to B. By identifying these similar entries, the matching efficiency of later steps may be increased.

In the first “filter” step in the matching process of the module, each identifier in gene set A is compared against each identifier in set B. If one entry in set A shares the same identifier and identifier type with one entry in set B, each entry is marked as an “ID match,” stored in a match list, and removed from each gene list. The gene entries that do not show any identifier match are passed to the next step.

For each of the gene entries that are passed from previous identifier match step, cluster identifiers are collected. This includes any common cluster identifier associated with a gene or with the identifiers of a gene. If one gene is assigned to more than one cluster in the same cluster system (e.g., the Unigene cluster system), this cluster identification information is considered contaminated, and the gene is passed onto the next step. After every valid cluster identifier is collected, a match is performed between set A and B by the algorithm of the module. Any match will be marked as a “cluster match,” stored in a match list, and removed from each gene list. The remaining entries are passed to the biomolecular sequence similarity match step.

In the final step, a sequence BLAST database maybe constructed for gene set B. A BLAST sequence similarity search is performed with the remaining sequences from set A as input by the algorithm of the module. A combination of statistical criteria including, but not limited to, blast similarity score, expectation value, match sequence length, and identity percentage, is used to judge if a gene in set A is a “BLAST match” to any gene in set B.

Various modifications and variations of the described methods and systems of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

The disclosures of all references and publications cited above are expressly incorporated by reference in their entireties to the same extent as if each were incorporated by reference individually.

We claim:

1. A method of comparing information contained in biomolecular databases comprising the steps of:
matching sequence identification information of biomolecular sequences in a first database with sequence identification information of biomolecular sequences in a second database, wherein any matched biomolecular sequences are placed into a matched sequence database;
matching biomolecular sequence information of biomolecular sequences in said first database with clusters of biomolecular sequences in said second database, wherein any matched biomolecular sequences are placed into said matched sequence database; and
matching complete biomolecular sequence information of biomolecular sequences in said first database with complete biomolecular sequence information of biomolecular sequences in said second database, wherein any matched biomolecular sequences are placed into said matched sequence database.

2. The method of claim 1, wherein said first database is an internal database.

3. The method of claim 2, wherein said first database comprises one or more databases selected from the group consisting of:
Incyte;
DNAchip Memo Status;
Gene Expression;
PRI Classification; and
Proteome.

4. The method of claim 1, wherein said second database is an external database.

5. The method of claim 4, wherein said second database comprises one or more databases selected from the group consisting of:
InterPro;
Ensembl;
dbSNP;
OMIM;
LocusLink;
GeneOntology;
UniGene; and
HomoloGene.

6. The method of claim 1, wherein said matching biomolecular sequence information of biomolecular sequences in said first database comprises matching with portions of a consensus or contig biomolecular sequence of biomolecular sequences in said second database.

7. The method of claims 1, wherein said first database is clustered prior to said first matching step.

8. The method of claim 1, wherein said matching steps are conducted when said second database is updated.

9. The method of claim 1, wherein said matching steps are conducted when said first database is updated.

10. The method of claim 1, wherein any matched biomolecular sequences are removed from said first database.

11. The method of claim 1, wherein any matched biomolecular sequences are removed from said second database.

12. A matched sequence database obtained from the method of claim 1.

13. A system for producing a matched sequence database comprising:
a first database with sequence identification information;
a second database with sequence identification information;
a first module adapted to match sequence identification information of biomolecular sequences with said first database with sequence identification information of biomolecular sequences with said second database, wherein any matched biomolecular sequences are placed into a matched sequence database and adapted to provide a modified first database and modified second database;
a second module adapted to match biomolecular sequence information of biomolecular sequences with said modified first database with clusters of biomolecular sequences in said modified second database, wherein any matched biomolecular sequences are placed into said matched sequence database; and
a third module adapted to match complete biomolecular sequence information of biomolecular sequences in said modified first database with complete biomolecular sequence information of biomolecular sequences in said modified second database, wherein any matched biomolecular sequences are placed into said matched sequence database.

14. The system of claim 13, wherein said first database is an internal database.

15. The system of claim 14, wherein said first database comprises one or more databases selected from the group consisting of:
Incyte;
DNAchip Memo Status;
Gene Expression;
PRI Classification; and
Proteome.

16. The system of claim 13, wherein said second database is an external database.

17. The system of claim 16, wherein said second database comprises one or more databases selected from the group consisting of:
InterPro;
Ensembl;
dbSNP;
OMIM;
LocusLink;
GeneOntology;
UniGene; and
HomoloGene.

18. The system of claim 13, wherein said matching biomolecular sequence information of biomolecular sequences in said first database comprises matching with portions of a consensus or contig biomolecular sequence of biomolecular sequences in said second database.

19. The system of claim 13, wherein said first database is clustered prior to said first matching step.

20. The system of claim 13, wherein said first, second, and third modules are executed when said second database is updated.

21. The system of claim 13, wherein said first, second, and third modules are executed when said first database is updated.

22. The system of claim 13, wherein any matched biomolecular sequences are removed from said first database.

23. The system of claim 13, wherein any matched biomolecular sequences are removed from said second database.

24. A method, in a computer system, for constructing a matched sequence database comprising the steps of:
matching sequence identification information of biomolecular sequences in a first database with sequence identification information of biomolecular sequences in a second database, wherein any matched biomolecular sequences are placed into a matched sequence database, resulting in a modified first and second database;
matching biomolecular sequence information of biomolecular sequences in said modified first database with clusters of biomolecular sequences in said modified second database, wherein any matched biomolecular sequences are placed into said matched sequence database; and
matching complete biomolecular sequence information of biomolecular sequences in said modified first database with complete biomolecular sequence information of biomolecular sequences in said modified second database, wherein any matched biomolecular sequences are placed into said matched sequence database.

25. The method of claim 24, wherein said first database is an internal database.

26. The method of claim 25, wherein said first database comprises one or more databases selected from the group consisting of:
Incyte;
DNAchip Memo Status;
Gene Expression;
PRI Classification; and
Proteome.

27. The method of claim 24, wherein said second database is an external database.

28. The method of claim 27, wherein said second database comprises one or more databases selected from the group consisting of:
InterPro;
Ensembl;
dbSNP;
OMIM;
LocusLink;
GeneOntology;
UniGene; and
HomoloGene.

29. The method of claim 24, wherein said matching biomolecular sequence information of biomolecular sequences in said first database comprises matching with portions of a consensus or contig biomolecular sequence of biomolecular sequences in said second database.

30. The method of claim 24, wherein said first database is clustered prior to said first matching step.

31. The method of claim 24, wherein said matching steps are conducted when said second database is updated.

32. The method of claim 24, wherein said matching steps are conducted when said first database is updated.

33. The method of claim 24, wherein any matched biomolecular sequences are removed from said first database.

34. The method of claim 24, wherein any matched biomolecular sequences are removed from said second database.

35. A computer program for constructing a matched sequence database comprising:
computer code providing an algorithm for matching sequence identification information of biomolecular sequences in a first database with sequence identification information of biomolecular sequences in a second database, wherein any matched biomolecular sequences are placed into a matched sequence database;
computer code providing an algorithm for matching biomolecular sequence information of biomolecular sequences in said modified first database with clusters of biomolecular sequences in said modified second database, wherein any matched biomolecular sequences are placed into said matched sequence database; and
computer code providing an algorithm for matching complete biomolecular sequence information of biomolecular sequences in said modified first database with complete biomolecular sequence information of biomolecular sequences in said modified second database, wherein any matched biomolecular sequences are placed into said matched sequence database.

36. The computer program of claim 35, wherein said first database is an internal database.

37. The computer program of claim 36, wherein said first database comprises one or more databases selected from the group consisting of:
Incyte;
DNAchip Memo Status;
Gene Expression;
PRI Classification; and
Proteome.

38. The computer program of claim 35, wherein said second database is an external database.

39. The computer program of claim 38, wherein said second database comprises one or more databases selected from the group consisting of:
InterPro;
Ensembl;
dbSNP;
OMIM;
LocusLink;
GeneOntology;
UniGene; and
HomoloGene.

40. The computer program of claim 35, wherein said matching biomolecular sequence information of biomolecular sequences in said first database comprises matching with portions of a consensus or contig biomolecular sequence of biomolecular sequences in said second database.

41. The computer program of claim 35, wherein said first database is clustered prior to said first matching step.

42. The computer program of claim 35, wherein said matching steps are conducted when said second database is updated.

43. The computer program of claim 35, wherein said matching steps are conducted when said first database is updated.

44. The computer program of claim 35, wherein any matched biomolecular sequences are removed from said first database.

45. The computer program of claim 35, wherein any matched biomolecular sequences are removed from said second database.

46. A computer system for providing users with the ability to access biomolecular sequence information from a matched sequence database comprising:
a computer processor;
a memory which is operatively coupled to said computer processor; and
a computer process stored in said memory which executes in said computer processor and which comprises:
a first module adapted to match sequence identification information of biomolecular sequences with a first database with sequence identification information of biomolecular sequences and with a second database, wherein any matched biomolecular sequences are stored in a matched sequence database located in said memory and adapted to provide a modified first database and a modified second database;
a second module adapted to match biomolecular sequence information of biomolecular sequences in said modified first database with clusters of biomolecular sequences in said modified second database, wherein any matched biomolecular sequences are stored in said matched sequence database located in said memory; and
a third module adapted to match complete biomolecular sequence information of biomolecular sequences in said modified first database with complete biomolecular sequence information of biomolecular sequences in said modified second database, wherein any matched biomolecular sequences are stored in said matched sequence database located in said memory.

47. The computer system of claim 46, wherein said first database is an internal database.

48. The computer system of claim 47, wherein said first database comprises one or more databases selected from the group consisting of:
Incyte;
DNAchip Memo Status;
Gene Expression;
PRI Classification; and
Proteome.

49. The computer system of claim 46, wherein said second database is an external database.

50. The computer system of claim 49, wherein said second database comprises one or more databases selected from the group consisting of:
InterPro;
Ensembl;
dbSNP;
OMIM;
LocusLink;
GeneOntology;
UniGene; and
HomoloGene.

51. The computer system of claim 46, wherein said matching biomolecular sequence information of biomolecular sequences in said first database comprises matching with portions of a consensus or contig biomolecular sequence of biomolecular sequences in said second database.

52. The computer system of claim 46, wherein said first database is clustered prior to said first matching step.

53. The computer system of claim 46, wherein said first, second, and third modules are executed when said second database is updated.

54. The computer system of claim 46, wherein said first, second, and third modules are executed when said first database is updated.

55. The computer system of claim 46, wherein any matched biomolecular sequences are removed from said first database.

56. The computer system of claim 46, wherein any matched biomolecular sequences are removed from said second database.

57. A computer process allowing a user to interactively access biomolecular sequence information from the matched sequence database of claim 10 comprising:
displaying query options for a biomolecular sequence information query accessing said matched sequence database; and
displaying results from said biomolecular sequence information query.

58. The computer process of claim 57 further comprising:
means for selecting one or more biomolecular sequences for which to display information.

59. The computer process of claim 57 further comprising:
a module adapted to select one or more external databases for which to display information related to said biomolecular sequence information from said matched sequence database.

60. The computer process of claim 57 further comprising:
a module adapted to select one or more fields of an external database for which to display information related to said biomolecular sequence information from said matched sequence database.

61. The computer process of claim 57 further comprising:
a module adapted to display information from one or more fields of one or more external databases related to said biomolecular sequence information from said matched sequence database.

62. A method of accessing biomolecular sequence information from a matched sequence database comprising:
selecting one or more biomolecular sequences for which to access biomolecular sequence information;
selecting one or more fields of said matched sequence database for which to retrieve biomolecular sequence information; and
performing a database query on said matched sequence database to retrieve said biomolecular sequence information.

63. A method comprising the step of providing the matched sequence database of claim 12 to a consumer.

64. The method of claim 63 further comprising the step of charging a fee to said consumer for providing said matched sequence database.

65. The method of claim 64, wherein said step of charging a fee to said consumer for providing said matched sequence database is selected from the group consisting of:
selling a license allowing access to said matched sequence database,
charging a per-access fee to said consumer for accessing said matched sequence database, and
charging a time-based fee to said consumer for accessing said matched sequence database.

66. A method comprising the step of providing the matched sequence database of claim 12 to a third party for access by a consumer.

67. A method comprising the step of providing a third party an interface by which said third party accesses the matched sequence database of claim 12.

68. The method of claim 67 further comprising the step of:
charging a fee paid by said third party for use of said matched sequence database.

69. The method of claim 68, wherein said charging a fee paid by said third party for use of said matched sequence database comprises one or more of the group consisting of:
a one-time fee;
a per-consumer fee; and
a time-based fee.

70. A method of providing a method to produce the matched sequence database of claim 12.

71. The method of claim 70 further comprising the step of purchasing an ability to use said matched sequence database.

72. The method of claim 71, wherein said step of purchasing the ability to use said matched sequence database comprises paying one or more of the group consisting of the following for the use of said matched sequence database:
a one-time fee;
a per-consumer fee; and
a time-based fee.

73. A microarray comprising one or more sequences or portions thereof, from the matched sequence database of claim 12.

74. A group of matched sequences selected from the matched sequence database of claim 12.
